(19) United States
(12) Patent Application Publication
(10) Pub. No.: US 2010/0173833 A1
(43) Pub. Date: Jul. 8, 2010

(54) METHODS AND COMPOSITION FOR USE OF CYCLIC ANALOGUES OF HISTATIN

(76) Inventors: Gilles Andre Lajoie, Waterloo (CA); Greg John Adams Vilk, London (CA); Dyanne Patricia Brewer, Orangeville (CA)

Correspondence Address: VALENTINE A COTTRILL;SUSAN TANDAN, 50 QUEEN STREET NORTH, STE. 1020, P.O. BOX 2248, KITCHENER, ON, N2H6M2, CA

(21) Appl. No.: 12/451,302

(22) PCT Filed: May 5, 2008
(86) PCT No.: PCT/CA2008/000853

§ 371 Date: Mar. 3, 2010

Related U.S. Application Data
(60) Provisional application No. 60/927,759, filed on May 5, 2007.

(51) Int. Cl. A61K 38/12; A61P 31/04; G01N 33/573; G01N 33/566

(52) U.S. Cl. 514/9; 435/7.4; 436/501

(57) ABSTRACT

Provided are novel compositions and therapeutic methods for treating bacterial and fungal disease with cyclic analogues of histatin. The cyclic analogues of histatin are advantageously more potent but less toxic than currently used anti-microbial agents. In addition, compositions comprising the cyclic analogue with other anti-microbial agents such as azole compounds are disclosed.

### FIELD OF THE INVENTION

The present invention relates to cyclic analogues of histatin, and to their use in novel compositions and methods of treatment.

### BACKGROUND OF THE INVENTION

Fungal and bacterial infections are a rising health risk as evidenced by the emergence of 9 million patients afflicted with these infections in the seven major markets worldwide. Patients at risk include those undergoing surgery, leukemic and HIV/AIDS patients and those who are immuno-compromised due to cancer, surgery or organ transplantation. This is partially as a result of the development of antibiotic-resistance in micro-organisms.

As a result, there is a growing unmet need in connection with anti-microbial agents, and in particular, anti-fungal and anti-bacterial agents, which represents a market opportunity to develop non-toxic anti-fungal agents with high potency in humans.

### SUMMARY OF THE INVENTION

Cyclic analogues of histatin have now been determined to be useful to treat anti-microbial infection in humans either alone or in combination with other antimicrobial agents. The cyclic analogues are advantageously more potent but less toxic than currently used anti-microbial agents. In addition, compositions comprising the cyclic analogue with other anti-microbial agents are synergistic, exhibiting greater than expected activity.

Thus, in one aspect of the present invention, there is provided a method of treating a microbial infection in a human, comprising administering to the human a therapeutically effective amount of a cyclic analogue of histatin.

In another aspect of the invention, an anti-microbial composition for use in treating a microbial infection in a human is provided comprising a cyclic analogue of histatin in combination with a pharmaceutically acceptable carrier.

In another aspect, a composition for treating a microbial infection is provided comprising a cyclic analogue of histatin and a second anti-microbial agent.

In further aspects, kits and articles of manufacture are provided. Kits comprise cyclic analogues of histatin as well as compliance means such as instructions for use. An article of manufacture in accordance with the invention comprises packaging within which is an anti-microbial composition comprising a cyclic analogue of histatin. The packaging is labelled to indicate that the composition is suitable for treating a microbial infection in a human.

In another aspect, a method of treating a microbial infection in a mammal is provided comprising administering to the mammal an anti-microbial agent in combination with a carrier molecule that targets microbial mitochondria.

In another aspect, a method of inhibiting microbial mycelial growth is provided comprising contacting a microorganism with a cyclic analogue of histatin.

In a further aspect, a method of treating a microbial infection in a mammal is provided comprising administering to the mammal a cyclic analogue of histatin that targets a protein selected from the group consisting of: microtubial-associated protein (YTM1), septin (CDC3), spindle dynamic control protein (SLK19), regulation of G-protein function (CRP1), HSP70 family member (SSA2), enolase I (ENO1), HSP member (SSE1), 26S proteosomal subunit (RPT5), HSP90, mitochondrial HSP protein (SSC1), beta1 subunit of ATPase complex (ATP2), mitochondrial aconitase/hydratase (ACO1) and microsomal ATPase (CDC48), RAV1, HSP60, dihydrolipoamide dehydrogenase (LPD1), 60S ribosomal subunit protein L3 (RPL3) and seryl-tRNA synthetase (SES1).

In another aspect, a method of screening candidate anti-microbial compounds is provided. The method comprises the steps of:

1) contacting a candidate compound with at least one target selected from the group consisting of: microtubial-associated protein (YTM1), septin (CDC3), spindle dynamic control protein (SLK19), regulation of G-protein function (CRP1), HSP70 family member (SSA2), enolase I (ENO1), HSP member (SSE1), 26S proteosomal subunit (RPT5), HSP90, mitochondrial HSP protein (SSC1), beta1 subunit of ATPase complex (ATP2), mitochondrial aconitase/hydratase (ACO1) and microsomal ATPase (CDC48), RAV1, HSP60, dihydrolipoamide dehydrogenase (LPD1), 60S ribosomal subunit protein L3 (RPL3) and seryl-tRNA synthetase (SES1); and

2) detecting whether or not said compound associates or interacts with one of said targets, wherein interaction with a target is indicative that said compound may have anti-microbial activity.

These and other aspects, features and advantages of the invention will become apparent from the following detailed description, claims and drawings.

### BRIEF DESCRIPTION OF THE DRAWINGS

FIG. 1 illustrates the amino acid sequences of several histatins;

FIG. 2 graphically illustrates the effect of temperature on the activity of a cyclic analogue of histatin (A) and the activity of 5 μM and 50 μM doses of the cyclic analogue (B);

FIG. 3 graphically illustrates the lack of toxicity of a cyclic histatin analogue on human primary cultured cells as compared with the toxicity of ketoconazole; and

FIG. 4 is a pie-chart illustrating the classes of microbial proteins with which a cyclic analogue of histatin associates.

### DETAILED DESCRIPTION

Provided are methods and compositions for use in treating microbial infections in humans comprising the administration of a cyclic analogue of histatin or a variant or derivative thereof.

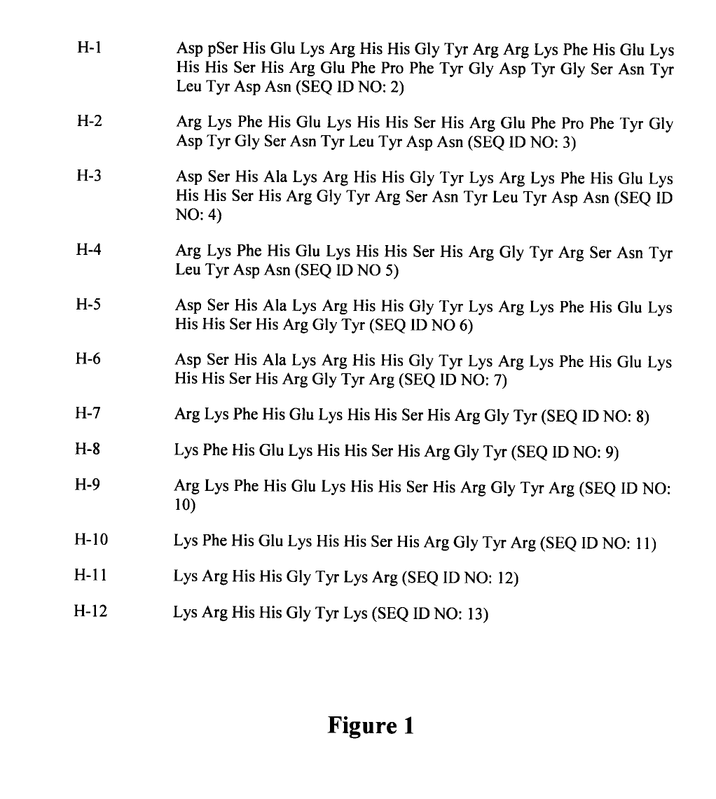

Cyclic analogues of histatin that may be used in the compositions and methods of the present invention are described and characterized in U.S. Pat. No. 6,555,650 and in Brewer, et al. (2002) Biochemistry 41:5526-5536, both of which are hereby incorporated by reference in their entireties. Generally, as used herein, the term “cyclic analogue of histatin” refers to cyclic analogues exhibiting anti-microbial activity that are formed from any histatin, or derivative or variant thereof, of suitable length to stably cyclize, including for example, histatin-1 (H-1) to histatin-12 (H-12), as set out in FIG. 1, particularly histatins H-1 to H-6, and more particularly, histatins H-1, H-3 and H-5. The term “variant” as it is used with respect to histatin, refers to a molecule that exhibits substantial sequence homology with a histatin, for example, at least about 60% homology, preferably at least about 80% homology, and more preferably at least about 90-95% homology. Examples of suitable variants include histatins in which lysine, glutamic acid or cysteine residues are introduced into the histatin to replace one or more existing amino acid residues in order to provide a variant that is readily cyclized to form a cyclic histatin analogue. The term “derivative” as used herein with respect to histatin refers to a histatin molecule that includes one or more modifications at a reactive site thereon, such as at a free carboxyl or amine group or other side chain group. Such modifications may be implemented in order to confer on the histatin analogue desirable properties such as increased stability, or improved cellular uptake. One cyclic histatin analogue in accordance with the invention is referred to herein as DB2121 and has the amino acid sequence, RHHCYKRKFHEKHHCHRGY (SEQ ID NO: 1).

The preparation of such cyclic analogues of histatin is described in U.S. Pat. No. 6,555,650, and generally involves standard methods of peptide synthesis, followed by amino acid substitutions, where desired, to facilitate cyclization. The linear histatin peptide is then cyclized under appropriate conditions for cyclization, as will be appreciated by one of skill in the art and as described in U.S. Pat. No. 6,555,650, which will depend on the amino acid residues involved in the cyclization reaction.

In accordance with a method of the invention, cyclic analogues of histatin have been determined to be useful to treat microbial infections, including for example bacterial infections and fungal infections. Thus, the present cyclic histatin analogues may be used to treat human bacterial infections caused by, for example, a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii, Helicobacter pylori or Chlamydia trachomitis. Non-limiting examples of diseases resulting from a bacterial infection in a human include otitis media, conjunctivitis, pneumonia, bacteremia, meningitis, sinusitis, pleural empyema and endocarditis, and meningitis, such as for example infection of cerebrospinal fluid.

The cyclic histatin analogues may also be used to treat fungal infections in humans caused by, for example, Cryptococcus spp., Candida spp., Aspergillus spp., Histoplasma spp., Coccidioides spp., Paracoccidioides spp. Blastomyces spp., Fusarium spp., Sporothrix spp., Trichosporon spp., Torulopsis spp., Rhizopus spp., Pseudallescheria spp., Dermatophytes spp., Paeciliomyces spp., Alternaria spp., Curvularia spp., Exophiala spp., Wangiella spp., Penicillium spp., Saccharomyces spp., Dematiaceous fungi and Pneumocystis carinii. Non-limiting examples of human disease resulting from a fungal infection include cryptococcal meningitis, athlete's foot, yeast infection, mold and mildew related illnesses, thrush, histoplasmosis, blastomycosis, onychomyosis and Tinea infections such as Tinea capitis, Tinea versicolor and Tinea pedis.

Therapeutically effective dosages of cyclic histatin analogues are administered to a human to treat a microbial infection. The term “therapeutically effective” as it is used herein with respect to dosages refers to a dosage that is effective to treat a given microbial infection without causing unacceptable adverse side effects. The term “administered” refers to any appropriate means of providing the cyclic histatin dosage to a recipient, and will depend on the dosage form being used as will be described. For example, the dosage may be administered orally, by injection, mucosally and topically as will be described in more detail. The term “treat” refers to at least partial inhibition of the microorganism causing the infection which may result in amelioration of one or more symptoms of the infection.

Therapeutically effective dosages according to the method, thus, are in the range of 0.01 ng to about 10 g per kg body weight, specifically in the range of about 1 ng to about 0.1 g per kg, and more specifically in the range of about 100 ng to about 10 mg per kg. However, as one of skill in the art will appreciate, the effective therapeutic dosage of the histatin cyclic analogues will vary depending on the symptoms, age and body weight of the patient being treated, the nature and severity of the infection to be treated or prevented and the route of administration. The present histatin analogues may be administered in a single dose or in divided doses.

The cyclic histatin analogues may be administered in the treatment of a microbial infection in a human alone or in a composition combined with a pharmaceutically acceptable adjuvant or carrier. The expression “pharmaceutically acceptable” means acceptable for use in the pharmaceutical arts, i.e. not being unacceptably toxic, or otherwise unsuitable for administration to a human. Examples of pharmaceutically acceptable adjuvants include, but are not limited to, diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule, lozenge, solution or suspension in an aqueous or non-aqueous liquid, an oil-in-water or water-in-oil liquid emulsion, an elixir or syrup are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, disintegrating agents, anti-oxidants, preservatives, colouring agents and flavouring agents may also be present. In another embodiment, the cyclic analogue may be formulated for application topically as a cream, lotion or ointment. For such topical application, the cyclic analogue is combined with an appropriate base such as a triglyceride base. Such creams, lotions and ointments may also contain a surface active agent and other cosmetic additives such as skin softeners and the like as well as fragrance. Aerosol formulations, for example, for nasal delivery, may also be prepared in which suitable propellant adjuvants are used. Compositions of the present invention may also be administered as a bolus, electuary, or paste. Compositions for mucosal administration are also encompassed, including oral, nasal, rectal or vaginal administration for the treatment of infections which affect these areas. Such compositions generally include one or more suitable non-irritating excipients or carriers comprising, for example, cocoa butter, polyethylene glycol, a suppository wax, a salicylate or other suitable carriers. Other adjuvants may also be added to the composition regardless of how it is to be administered which, for example, may aid to extend the shelf-life thereof.

Thus, the present cyclic histatin analogues may be included in compositions in which an anti-microbial is advantageous such as medicines to treat particular infections as described above, other healthcare products such as wound treatments, eye drops, toothpaste, mouth washes or rinses, soaps, bath gel, shampoo, body lotion and beauty products such as makeup or other cosmetics.

In another aspect, the present cyclic analogues of histatin may be administered to a mammal in need of treatment for an microbial infection combined with another active ingredient, for example, a second antimicrobial agent such as an antifungal or antibacterial agent. Antibacterial agents that may be used as a second antimicrobial agent in the compositions and methods of the present invention include, but are not limited to, cephalosporins including cephalosporins I generation such as Cefadroxil, Cefazolin, Cephalexin, Cephalothin, Cephapirin, and Cephradine; cephalosporins II generation such as Cefaclor, Cefamandol, Cefonicid, Cefotetan, Cefoxitin, Cefprozil, Ceftmetazole, Cefuroxime, Cefuroxime axetil, and Loracarbef; cephalosporins III generation such as Cefdinir, Ceftibuten, Cefditoren, Cefetamet, Cefpodoxime, Cefprozil, Cefuroxime (axetil), Cefuroxime (sodium), Cefoperazone, Cefixime, Cefotaxime, Cefpodoxime proxetil, Ceftazidime, Ceftizoxime, and Ceftriaxone; and cephalosporins IV generation such as Cefepime; quinolones and fluoroquinolones such as Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, and Pefloxacin; penicillins such as Amoxicillin, Ampicillin, Bacampicillin, Carbenicillin Indanyl, Mezlocillin, Piperacillin, and Ticarcillin; penicillin/beta lactamase inhibitors such as Amoxicillin-Clavulanic Acid, Ampicillin-Sulbactam, Benzylpenicillin, Cloxacillin, Dicloxacillin, Methicillin, Oxacillin, Penicillin G (Benzathine, Potassium, Procaine), Penicillin V, Piperacillin+Tazobactam, Ticarcillin+Clavulanic Acid, and Nafcillin; carbepenems such as Imipenem-Cilastatin and Meropenem; a monobactam such as Aztreonam; macrolides and lincosamines such as Azithromycin, Clarithromycin, Clindamycin, Dirithromycin, Erythromycin, Lincomycin, and Troleandomycin; glycopeptides such as Teicoplanin and Vancomycin; rifampins such as Rifabutin, Rifampin, and Rifapentine; oxazolidonones such as Linezolid; tetracyclines such as Demeclocycline, Doxycycline, Methacycline, Minocycline, Oxytetracycline, Tetracycline, and Chlortetracycline; aminoglycosides such as Amikacin, Gentamicin, Kanamycin, Neomycin, Netilmicin, Streptomycin, Tobramycin, and Paromomycin; streptogramins such as Quinopristin+Dalfopristin; sulfonamides such as Mafenide, Silver Sulfadiazine, Sulfacetamide, Sulfadiazine, Sulfamethoxazole, Sulfasalazine, Sulfisoxazole, Trimethoprim-Sulfamethoxazole, and Sulfamethizole; and other antibiotic agents such as Bacitracin, Chloramphenicol, Colistemetate, Fosfomycin, Isoniazid, Methenamine, Metronidazol, Mupirocin, Nitrofurantoin, Nitrofurazone, Novobiocin, Polymyxin B, Spectinomycin, Trimethoprim, Colistin, Cycloserine, Capreomycin, Pyrazinamide, Para-aminosalicyclic acid, and Erythromycin ethylsuccinate+sulfisoxazole.

Examples of antifungal agents that may be used as the second antimicrobial agent in the compositions and methods of the present invention include, but are not limited to, azoles such as fluconazole, voriconazole, clotrimazole, itraconazole, ketoconazole, miconazole, ER 30346, SCH 56592; polyenes such as amphotericin B, nystatin or liposomal and lipid forms thereof such as Abelcet, AmBisome and Amphocil; purine or pyrimidine nucleotide inhibitors such as flucytosine; or polyoxins such as nikkomycins, in particular nikkomycin Z or other chitin inhibitors, elongation factor inhibitors such as sordarin and analogs thereof, mannan inhibitors such as predamycin, bactericidal/permeability-inducing (BPI) protein products such as XMP.97 or XMP.127.

The cyclic histatin analogue and the second antimicrobial agent may be administered to a mammal in the treatment of an infection either separately, or in combination. The term “mammal” is used herein to encompass both human and non-human mammals.

In addition, the cyclic histatin analogue may be combined with a second anti-microbial agent to provide an effective anti-microbial composition. The composition may additionally include pharmaceutically acceptable adjuvants to facilitate the administration thereof in a selected dosage form as described above. The amount of cyclic histatin analogue combined with the second antimicrobial agent will vary with the cyclic histatin analogue being used as well as the second antimicrobial agent being used, as will be appreciated by one of skill in the art. Generally, the amount of each will be an amount suitable for administration to a mammal, as determined using appropriate studies, that is not toxic or otherwise unacceptable. Thus, the dosage of cyclic histatin in the composition may be present in the range of 0.01 ng to about 10 g per kg body weight, while the dosage of the second antimicrobial agent will be present in a suitable dosage range as determined for that agent. In a preferred embodiment, the combination of a cyclic histatin analogue and another anti-microbial agent provides a composition that yields anti-microbial activity that is greater than the expected anti-microbial effect thereof, i.e. greater than the expected additive effect of the combination, and thus, is a synergistic composition. Accordingly, the dosages of each of the cyclic histatin and the second antimicrobial agent in the combined composition may advantageously be less than the dosage generally administered when the cyclic histatin and the second antimicrobial are used alone. This is particularly desirable to reduce toxicity and other undesirable affects that may be present when using full-strength dosages of antimicrobial agents.

In another aspect of the invention, kits are provided comprising an anti-microbial composition that includes a cyclic analogue of histatin in combination with a pharmaceutically acceptable adjuvant or carrier, and compliance means such as instructions for its use to treat a microbial infection in a human. The instructions may additionally include an indication of recommended dosages to be used. Additional compliance means may also be included in such kits including any means which facilitate the correct usage of the composition. Such compliance means may include additional instructions, dispensing means, and the like. Alternatively, an article of manufacture is provided comprising packaging within which is contained the anti-microbial composition. In this case, the packaging is labeled at least to indicate that the composition is suitable for treating a microbial infection in a human, and may be further labeled to indicate recommended dosages and other conditions for use. In accordance with preceding aspects of the invention, the kits and articles of manufacture may additionally comprise a second antimicrobial agent either combined with the cyclic histatin analogue or separate thereform.

In another aspect of the present invention, methods of screening compounds for use as anti-microbial agents are provided that are based on the determination that certain microbial proteins are targets of cyclic analogues of histatin. These protein targets include microtubial-associated protein (YTM1), septin (CDC3), spindle dynamic control protein (SLK19), regulation of G-protein function (CRP1), HSP70 family member (SSA2), enolase I (ENO1), HSP member (SSE1), 26S proteosomal subunit (RPT5), HSP90, mitochondrial HSP protein (SSC1), beta1 subunit of ATPase complex (ATP2), mitochondrial aconitase/hydratase (ACO1) and microsomal ATPase (CDC48), RAV1, HSP60, dihydrolipoamide dehydrogenase (LPD1), 60S ribosomal subunit protein L3 (RPL3) and seryl-tRNA synthetase (SES1). The systemics name of certain of these proteins from the Candida genome database (http://www.candidagenome.org) are as follows: YTM1-orf19.4815; cdc3-orf19.1055; SLK19-orf19.6763; CRP1-orf19.4784; SSA2-orf19.1065; ENO1-orf19.395; SSE1-orf19.2435; SSC1-orf19.1896; ATP2-orf19.5653; ACO1-orf19.6385; and CDC48-orf19.2340

The method of screening candidate anti-microbial agents includes incubating the candidate compound with one or more of said target proteins, either per se or in a whole cell environment, and determining whether or not the candidate associates with, e.g. including via covalent, electrostatic, hydrophobic, aromatic, ionic and dipolar associations, via hydrogen donating and accepting forces, or otherwise interacts with, the target. The determination of an association with a target protein is indicative that the candidate may have anti-microbial activity, and represents a candidate for further testing including, for example, microbial cell inhibition studies. Alternatively, it may be determined whether or not the candidate modulates the activity of the protein target using an appropriate assay for this purpose as would be appreciated by one of skill in the art. A determination that the candidate modulates, either by inhibition or activation, a target protein is indicative that the candidate may have anti-microbial activity and therefore warrants further study. Although the candidate anti-microbial agents suitable for screening may be any selected compound, cyclic analogues of histatin are particularly suitable for screening in this manner.

The identification of the foregoing protein targets for cyclic analogues of histatin also provides a further method of treating a microbial infection in a mammal. The method includes the steps of administering an anti-microbial agent in combination with a carrier molecule that targets a protein selected from the group consisting of: microtubial-associated protein (YTM1), septin (CDC3), spindle dynamic control protein (SLK19), regulation of G-protein function (CRP1), HSP70 family member (SSA2), enolase I (ENO1), HSP member (SSE1), 26S proteosomal subunit (RPT5), HSP90, mitochondrial HSP protein (SSC1), beta1 subunit of ATPase complex (ATP2), mitochondrial aconitase/hydratase (ACO1) and microsomal ATPase (CDC48), RAV1, HSP60, dihydrolipamide dehydrogenase (LPD1), beta1 subunit of ATPase complex (ATP2), 60s ribosomal subunit protein L3 (RPL3) and seryl-tRNA synthetase (SES1). Although the carrier molecule may be any molecule suitable to associate with one or more of the target proteins, cyclic analogues of histatin are particularly suitable for this purpose.

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the claims that follow.

Embodiments of the invention are described by reference to the following specific examples which are not to be construed as limiting.

### Example 1

### Activity of DB2121 Against Fungi and Bacteria

The efficacy of the cyclic analogue, DB2121, to inhibit cell growth was measured in various fungal and bacterial organisms and the results were compared to several other known anti-fungal compounds such as ketoconazole (Table 1). The MIC of DB2121 in fungal or yeast strains was at least 100-fold more potent than either histatin H5 or the previously developed patented analogue, P-113. DB2121 was found to possess an approximate 40-fold greater ability over histatin H5 to inhibit the Gram-positive Bacillus subtilis.

TABLE 1

| | DRUG (uM MIC) | | | | | | |
|---|---|---|---|---|---|---|---|
| ORGANISM | DB2121 | H5 | P113 | FLU | ITRA | KETO | AMP-b |
| FUNGI | | | | | | | |
| (Lab Strains) | | | | | | | |
| Candida albicans (SC5314) | 4.5 | >350 | >350 | ≧97.9 | 3.1 | 5.5 | 1.2 |
| (Clinical Strains) | | | | | | | |
| Candida albicans (isolate #3) | 8.0 | >350 | >350 | ≧97.9 | ≧7.09 | ≧9.41 | 0.6 |
| Candida albicans (isolate #4) | 4.5 | >350 | >350 | ≧97.9 | ≧7.09 | ≧9.41 | 4.5 |
| Candida albicans (isolate #5) | 4.5 | >350 | >350 | ≧97.9 | ≧7.09 | ≧9.41 | 3.2 |
| Candida albicans (isolate #11) | 6.5 | >350 | >350 | ≧97.9 | 5.5 | 3.4 | ≦0.625 |
| Candida albicans (isolate #28) | 4.5 | >350 | >350 | ≧97.9 | ≧7.09 | ≧9.41 | ≦0.625 |
| Candida albicans (isolate #77) | 4.5 | >350 | >350 | ≧97.9 | ≧7.09 | ≧9.41 | (N/D) |
| Candida albicans (isolate #98) | 4.5 | >350 | >350 | ≧97.9 | ≧7.09 | 6.2 | ≦0.625 |
| Candida albicans (isolate #99) | 4.5 | >350 | >350 | ≧97.9 | ≧7.09 | 6.2 | 5.5 |
| Candida albicans (isolate #AST97 | 7.8 | >350 | >350 | ≧97.9 | 5.4 | 1.2 | 4.5 |
| Candida dubliniensis (isolate #10) | 4.5 | >350 | >350 | ≧97.9 | ≧7.09 | ≧9.41 | 5.4 |
| Candida tropicalis (isolate #93) | 4.5 | >350 | >350 | ≧97.9 | ≧7.09 | ≧9.41 | ≧6.5 |
| BACTERIA | | | | | | | |
| (Lab Strains) | | | | | | | |
| S. aureus (ATCC 29213) | 12.5 | (N/D) | (N/D) | (N/D) | (N/D) | (N/D) | (N/D) |
| S. haemolyticus (ATCC 29970) | 4.5 | (N/D) | (N/D) | (N/D) | (N/D) | (N/D) | (N/D) |
| S. aurens (ATCC 43300) | 5.5 | (N/D) | (N/D) | (N/D) | (N/D) | (N/D) | (N/D) |
| S. aurens (ATCC 25923) | 4.5 | (N/D) | (N/D) | (N/D) | (N/D) | (N/D) | (N/D) |
| Bacillus subtilis | 23.9 | >350 | >350 | (N/D) | (N/D) | (N/D) | (N/D) |
| (Clinical Strains) | | | | | | | |
| MRSAS. aureus (STRAIN 1) | 11.5 | >350 | >350 | (N/D) | (N/D) | (N/D) | (N/D) |
| MRSAS. aureus (STRAIN 2) | 11.5 | >350 | >350 | (N/D) | (N/D) | (N/D) | (N/D) |

1) H5 = Histaitn H5, KETO = Ketoconazole, FLU = Fluconazole, ITRA = Itraconazole, AMP-b = Amphoteracin-h, P113 (former Demegen product), MIC = Minimum Inhibitory concentration
2) All incubations were performed (a) 37° C. for one hour in 10 mM NaCl/50 mM MOPS, pH 7.2. After this time period, aliqouts were spiked into YPD broth for determination of MIC after 24 hours of growth @ 30° C. (fungi) or 37° C. (bacteria)
3) Clinical Candida isolates were oral swabs or a blood culture (AST97) from HIV-infected patients

### Example 2

### Activity in Combination with Another Anti-Microbial Agent

In similar cell inhibition studies as those described in Example 1 above, DB2121 was also shown to kill Candida albicans strains when used in combination with other anti-microbial agents, including ketoconazole, itraconazole and flucoconanzole.

Generally, a logarithmatically grown culture of Candida albicans was washed twice in a solution containing 20 mM Tris-Cl, pH 7.2 and 20 mM NaCl and then suspended in this solution. Various concentrations of cyclic Histatin analogue and either fluconazole, itraconazole or ketoconazole were added immediately to this culture and shaken vigorously at 37° C. for 1 hour. Aliquots were spiked at a dilution of 1:100 into pre-warmed YPD culture media in 24-well dishes. The dishes were incubated in a 37° incubator for a period of 24 hours to monitor growth and determine the Minimum Inhibitory Concentration (MIC) required for each compound either separately as controls and in combination.

Table 2 indicates that in a combination therapy using cyclic histatin analogue and either of the three tested antimicrobial compounds, concentrations of up to 12-fold less of cyclic histatin analogue and also the additional antimicrobial compounds were required compared to the drugs when administered separately.

TABLE 2

| TREATMENT | MIC (DB2121^{a}) μM | MIC (azole^{b}) μM |
|---|---|---|
| DB2121 | 11 | N/A |
| DB2121 + Fluconazole | 0.375 | 8.15 |
| DB2121 + Ketoconazole | 0.375 | 0.078 |
| DB2121 + Itraconazole | 0.375 | 0.059 |
| Fluconazole | N/A | 97.8 |
| Ketoconazole | N/A | 0.94 |
| Itraconazole | N/A | 0.71 |

^{a}refers to cyclic Histatin analogue
^{b}refers to Fluconazole, Ketoconazole or Itraconazole

For these specific combinations, a suitable working range of each of the cyclic histatin and the second antimicrobial agent would be as follows:

i) an amount of cyclic Histatin analogue of equal to or greater than 0.37 uM used with ketoconazole in an amount of equal to or greater than 0.078 uM

ii) an amount of cyclic Histatin analogue of equal to or greater than 0.37 uM used with fluconazole in an amount of equal to or greater than 8.15 uM

iii) an amount of cyclic Histatin analogue of equal to or greater than 0.37 uM used

with itraconazole in an amount of equal to or greater than 0.059 uM

### Example 3

### Inhibition of Microbial Mycelial Growth

C. albicans (SC5314) fungi were cultured according to standard methods. Once in log phase, the fungal cells were resuspended in 10 mM sodium phosphate and incubated for 1.5 hours at 37° C. in the presence of either (A) 50 μM ketoconazole, (B) vehicle, (C) 50 μM Histatin H5, or (D) 50 μM DB2121. An aliquot of the mixture was then spiked into YPD containing 10% FBS and cultured at 37° C. At various time points, the cells were imaged under phase contract microscopy at 40× magnification. The experiment was performed at last three times.

A significant inhibition of mycelial growth was noted when C. albicans was pre-incubated with DB2121. Moreover, DB2121 was effective to inhibit mycelial growth pre- and post-hyphae (data not shown) induction. Serial dilutions indicated that mycelial growth in the presence of DB2121 was approximately 500-1000 fold less than control and with other treatments, such as exposure to ketoconazole or histatin H5.

### Example 4

### Conditions of Inhibition of by DB2121

C. albicans (SC5314) were cultured according to standard procedures. Once in log phase, the fungal cells were resuspended in 10 mM sodium phosphate and the DB2121 peptide was tested for efficacy against C. albicans under various conditions. DB2121 was added to the fungal cells and incubated at either 4° C. or 37° C. for 1.5 hours with shaking. In a separate experiment, C. albicans was incubated with either 5 or 50 μM DB2121 (f.c.) for a total of 1.5 hours at 37° C. At certain time points, samples were taken to monitor cell viability. In a related set of experiments, (1) vehicle or (2, 3) Dansylated DB2121 was introduced to C. albicans under the conditions stated above. Following exposure, the fungal cells were counter-stained with SYTOX Green reagent to monitor for cell viability. The experiments were performed at least three times with similar results. Error bars represent standard deviation.

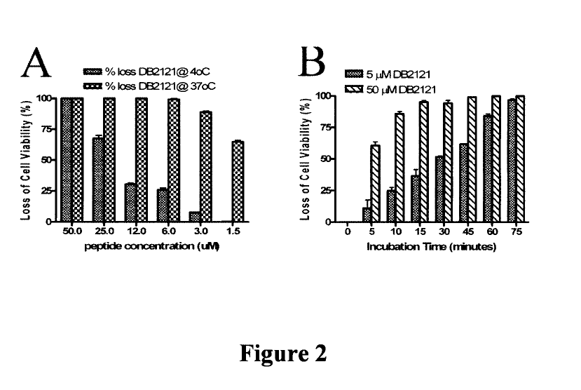

The activity of DB2121 was determined to be temperature-dependent, and in particular, more potent at physiological temperatures (FIG. 2A) and acts quickly to kill Candida in as little as 5 minutes of exposure at certain concentrations (e.g. 50 μM) (FIG. 2B).

### Example 5

### Toxicology of DB2121

Toxicological studies of DB2121 in human cells were also undertaken. Primary human neonatal foreskin epithelial cells were used to examine DB2121 toxicity, using a method as described in Min et al. 2004. Nat Biotechnol 22:717-23, the contents of which are incorporated herein by reference. Briefly, when the cells were in log phase, (A) 50 μM DB2121, B) 50 μM Histatin H5, (C) 50 μM ketoconazole, or (D) DMSO was added to the culture medium. At 18 and 36 hours following addition of compound, the foreskin epithelial cells were imaged using phase contract microscopy at 20× magnification. (E) Immediately following the image analysis, proteins were extracted, separated on SDS-PAGE gels, Western blotted, and probed for Phosphorylated and non-phosphorylated ERK 1, 2 to monitor the proliferation of the foreskin keratinocytes. The experiments was repeated three times.

Following the foregoing treatment, the human primary cells appeared morphologically normal except when treated with ketoconazole. The toxicity of ketoconazole to mammalian cells has been noted previously.

It was then investigated whether or not these phenotypes were reflected at the molecular level. The primary cells were lysed and monitored for a marker of proliferation namely phosphorylated ERK 1, 2. Significant inhibition of ERK 1, 2 activation in the ketoconazole-treated cells was observed as compared to control. Conversely, activated ERK 1, 2 was not suppressed in DB2121-treated cells as compared to controls.

Both studies show that DB2121 is non-toxic to human primary cells at working (e.g. antimicrobial) concentrations of 50 μM.

To determine range of non-toxic concentrations of DB2121, various concentration s of DB2121, 50 μM ketoconazole or DMSO as a control were separately added to primary human foreskin epithelial cell culture. At 0, 36 and 72 hours after addition, the epithelial cells were titriated off the culture plate and the viable cell number was determined. The experiment was performed in triplicate. At 72 hours, the foreskin epithelial cells exposed to the various compounds were imaged at 20× magnification using phase contrast microscopy. For cell counting, the epithelial cells were stained with trypan exclusion stain to monitor cell viability.

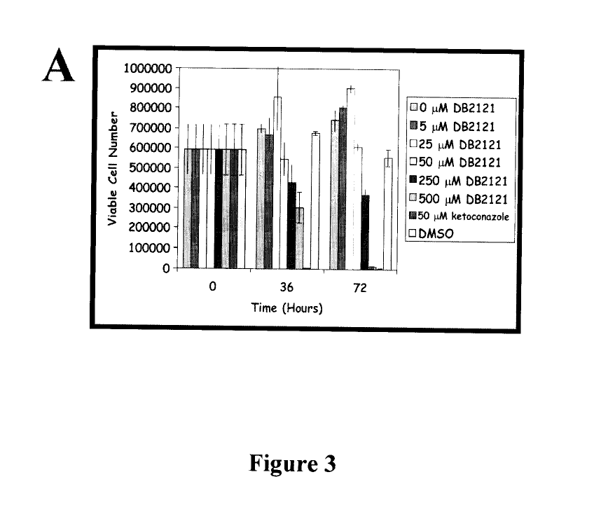

These results show minimal toxicity of DB2121 in primary cultured cells and a superior toxicity profile when compared to ketoconazole (FIG. 3).

In addition, mice were able to withstand a concentration of 15 mg/kg of DB2121 when injected via an intra-peritoneal route. When injected into adult rats, a concentration of up to and including 1.5 mg/kg caused no harm when injected intra-venously.

### Example 6

### In Vitro Targets of DB2121

Confocal microscopy using fluorescent-tagged versions of DB2121 determined that DB2121 does enter Candida albicans (SC5314) and that it locates specifically within the mitochondria of the organism (as determined by fluorescent probes specific for yeast).

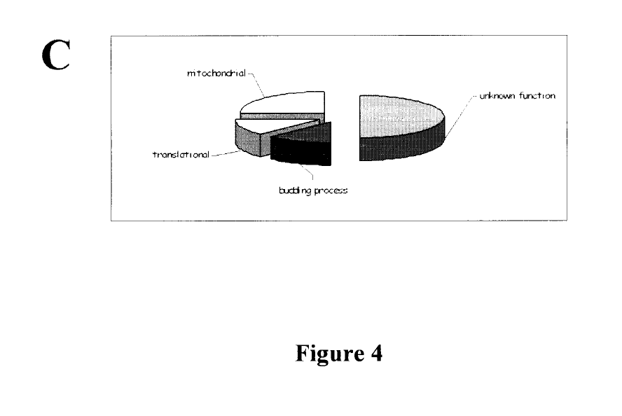

In an attempt to elucidate the mode of action of DB2121, Candida albicans was cultured in liquid media to an OD600 of ˜0.40-0.80 using standard protocols. At this point, the cells were harvested and the whole cell extract was collected. Equal amounts of protein were passed over glutathione-agarose columns containing purified GST or GST-DB2121. The column was extensively washed and the bound proteins were eluted with a gradient of free glutathione. Fractions were collected and the proteins from these fractions were resolved on 12% SDS-PAGE gels. The gels were stained. To identify the proteins of interest, the protein extracted, dried down and taken up in 0.2% formic acid. The peptides obtained from each protein band were then analyzed using LC/MS/MS on a Micromass. A pie chart indicating the classes of proteins identified in Candida albicans is shown in FIG. 4.

The DB2121 protein targets identified in Candida albicans (SC5314) were: microtubial-associated protein (YTM1), septin (CDC3), spindle dynamic control protein (SLK19), regulation of G-protein function (CRP1), HSP70 family member (SSA2), enolase I (ENO1), HSP member (SSE1), 26S proteosomal subunit (RPT5), HSP90, mitochondrial HSP protein (SSC1), beta1 subunit of ATPase complex (ATP2), mitochondrial aconitase/hydratase (ACO1) and microsomal ATPase (CDC48), RAV1, HSP60, dihydrolipoamide dehydrogenase (LPD1), 60S ribosomal subunit protein L3 (RPL3) and seryl-tRNA synthetase (SES1). Generally, the proteins listed above belong to different functional groups—proteins associated with morphogenesis/cell cycle/cell spindle/cytokinesis, proteins associated with the mitochondria, proteins associated with cell stress and metabolism, and proteins associated with translation control. These data are in good correlation with the data obtained from confocal microscopy.

### Example 7

### Stability of DB2121

DB2121 cyclic analogue was determined to be stable in vitro in human saliva for at least 72 hours as determined by mass spectrometry. The cyclic analogue was incubated in human saliva at a concentration of 1 uM and at a temperature of 37° C. in vitro. At various time points from 0 to 72 hours, 1.0 ul aliquots were taken and injected directly into a Micromass Quattro Micro mass spectrometer. Data was collected for a total of 3 minutes. The data was then processed using Mass Lynx 4.0 Analysis software.

The expected average mass of the cyclic histatin analogue in its active form is expected to be 2557.93. Cyclic histatin analogue was shown to be present in the human saliva up to at least 72 hours incubation. The peaks within an acceptable error of approximately 1 mass unit corresponding to cyclic histatin analogue were 2556.95 at 0 hours, 2557.65 at 24 hours, and 2557.10 at 72 hours incubation.

We claim:

1. A method of treating a microbial infection in a human, comprising administering to the human a therapeutically effective amount of a cyclic analogue of histatin.

2. The method of claim 1, wherein the cyclic analogue of histatin is a cyclic analogue of histatin H5.

3. The method of claim 1, wherein the cyclic analogue is prepared from a histatin in which at least one of the histatin amino acids is substituted with an amino acid selected from the group consisting of glutamic acid, lysine, cysteine and other thiol-containing amino acids to permit cyclization of the histatin.

4. The method of claim 2, wherein the cyclic analogue has the sequence of RHHCYKRKFHEKHHCHRGY (SEQ ID No. 1).

5. The method of claim 1, wherein the microbial infection is a bacterial infection.

6. The method of claim 1, wherein the microbial infection is a fungal infection.

7. The method of claim 1, further comprising administering an effective dose of a second anti-microbial agent.

8. The method of claim 7, wherein the anti-microbial agent is selected from the group consisting of an anti-fungal agent and an anti-bacterial agent.

9. The method of claim 7, wherein the anti-microbial agent is an azole compound.

10. The method of claim 9, wherein the azole compound is selected from the group consisting of fluconazole, voriconazole, clotrimazole, itraconazole, ketoconazole and miconazole.

11. An anti-microbial composition suitable for treating disease in a human resulting from infection by a microorganism comprising a cyclic analogue of histatin and at least one pharmaceutically acceptable carrier.

12. The composition as defined in claim 11, wherein the infection is selected from the group consisting of a bacterial infection and a fungal infection.

13. The composition as defined in claim 11, comprising a second anti-microbial agent.

14. The composition of claim 13, wherein the anti-microbial agent is an azole compound.

15. The composition of claim 14, wherein the azole compound is selected from the group consisting of fluconazole, voriconazole, clotrimazole, itraconazole, ketoconazole and miconazole.

16. A composition as defined in claim 11, wherein the cyclic analogue of histatin is selected from the group consisting of a cyclic analogue of H1, H3 and H5.

17. A composition as defined in claim 16, wherein the cyclic analogue is a cyclic analogue of histatin H5.

18. A composition as defined in claim 17, wherein the cyclic analogue has the sequence, RHHCYKRKFHEKHHCHRGY (SEQ ID No. 1).

19. An anti-microbial composition, comprising a cyclic analogue of histatin in combination with a second anti-microbial agent.

20. A composition as defined in claim 19, wherein the cyclic analogue of histatin is selected from the group consisting of a cyclic analogue of H1, H3 and H5.

21. A composition as defined in claim 20, wherein the cyclic analogue is a cyclic analogue of histatin H5.

22. A composition as defined in claim 21, wherein the cyclic analogue has the sequence, RHHCYKRKFHEKHHCHRGY (SEQ ID No. 1).

23. A composition as defined in claim 16, wherein the anti-microbial agent is an azole compound.

24. A composition as defined in claim 23, wherein the azole compound is selected from the group consisting of fluconazole, voriconazole, clotrimazole, itraconazole, ketoconazole and miconazole.

25. An article of manufacture comprising packaging within which is an anti-microbial composition comprising a cyclic analogue of histatin, wherein said packaging is labelled to indicate that the composition is suitable for treating a microbial infection in a human.

26. A method of treating an anti-microbial infection in a mammal, comprising administering to the mammal an anti-microbial agent in combination with a carrier molecule that targets microbial mitochondria.

27. A method as defined in claim 26, wherein the carrier molecule is a cyclic analogue of histatin.

28. A method as defined in claim 27, wherein the cyclic analogue is an analogue of one of histatin H1, H3 and H5.

29. A method as defined in claim 28, wherein the cyclic analogue is an analogue of H5.

30. A method as defined in claim 29, wherein the analogue has the sequence, RHHCYKRKFHEKHHCHRGY.

31. A method of inhibiting microbial mycelial growth in a mammal comprising administering to the mammal a cyclic analogue of histatin.

32. A method of treating a microbial infection in a mammal, comprising administering to the mammal a cyclic analogue of histatin that targets a protein selected from the group consisting of: microtubial-associated protein (YTM1), septin (CDC3), spindle dynamic control protein (SLK19), regulation of G-protein function (CRP1), HSP70 family member (SSA2), enolase I (ENO1), HSP member (SSE1), 26S proteosomal subunit (RPT5), HSP90, mitochondrial HSP protein (SSC1), beta1 subunit of ATPase complex (ATP2), mitochondrial aconitase/hydratase (ACO1) and microsomal ATPase (CDC48), RAV1, HSP60, dihydrolipoamide dehydrogenase (LPD1), 60S ribosomal subunit protein L3 (RPL3) and seryl-tRNA synthetase (SES1).

33. A method of screening candidate anti-microbial compounds comprising the steps of:
(a) contacting a candidate compound with at least one target selected from the group consisting of: microtubial-associated protein (YTM1), septin (CDC3), spindle dynamic control protein (SLK19), regulation of G-protein function (CRP1), HSP70 family member (SSA2), enolase I (ENO1), HSP member (SSE1), mitochondrial HSP protein (SSC1), beta1 subunit of ATPase complex (ATP2), mitochondrial aconitase/hydratase (ACO1) and microsomal ATPase (CDC48); and
(b) detecting whether or not said compound associates with said target, wherein detection of an association between the compound and the target is indicative that said compound may have anti-microbial activity.

34. A method as defined in claim 33, wherein the candidate compound is a cyclic analogue of histatin

35. A kit, comprising a composition comprising a cyclic analogue of histatin and instructions for use in the treatment of infection in a human.


SEQUENCE LISTING (13 sequences)

SEQ ID NO: 1 - protein, 19 aa, Artificial
  cyclic analogue of histatin
RHHCYKRKFHEKHHCHRGY

SEQ ID NO: 2 - protein, 38 aa, Human
DSHEKRHHGYRRKFHEKHHSHREFPFYGDYGSNYLYDN

SEQ ID NO: 3 - protein, 27 aa, Human
RKFHEKHHSHREFPFYGDYGSNYLYDN

SEQ ID NO: 4 - protein, 32 aa, Human
DSHAKRHHGYKRKFHEKHHSHRGYRSNYLYDN

SEQ ID NO: 5 - protein, 21 aa, Human
RKFHEKHHSHRGYRSNYLYDN

SEQ ID NO: 6 - protein, 24 aa, Human
DSHAKRHHGYKRKFHEKHHSHRGY

SEQ ID NO: 7 - protein, 25 aa, Human
DSHAKRHHGYKRKFHEKHHSHRGYR

SEQ ID NO: 8 - protein, 13 aa, Human
RKFHEKHHSHRGY

SEQ ID NO: 9 - protein, 12 aa, Human
KFHEKHHSHRGY

SEQ ID NO: 10 - protein, 14 aa, Human
RKFHEKHHSHRGYR

SEQ ID NO: 11 - protein, 13 aa, Human
KFHEKHHSHRGYR

SEQ ID NO: 12 - protein, 8 aa, Human
KRHHGYKR

SEQ ID NO: 13 - protein, 7 aa, Human
KRHHGYK